(19) United States
(12) Patent Application Publication
(10) Pub. No.: US 2026/0272814 A1
(43) Pub. Date: Sep. 17, 2026

(54) COMPOSITION FOR COLOURING FIBRES

(71) Applicant: VIVIDYE AB, Göteborg (SE)

(72) Inventors: Romain BORDES, Göteborg (SE); Shana PINSON, Göteborg (SE); Anna-Karin HELLSTRÖM, Göteborg (SE); Roujin GHAFFARI, Göteborg (SE); Kenneth GACUTNO ARANDIA, Göteborg (SE); Emma SIMON, Göteborg (SE); Viktor ERIKSSON, Göteborg (SE); Emman INGO, Göteborg (SE)

(73) Assignee: VIVIDYE AB, Göteborg (SE)

(21) Appl. No.: 19/475,534

(22) PCT Filed: Apr. 15, 2024
(86) PCT No.: PCT/SE2024/050357

§ 371 Date: Oct. 15, 2025

(30) Foreign Application Priority Data
Apr. 17, 2023 (SE) 2350454-1

(51) Int. Cl. A61K 8/84; A61K 8/04; A61K 8/49; A61Q 5/10

(52) U.S. Cl. CPC A61K 8/84; A61K 8/04; A61K 8/49; A61Q 5/10; A61K 2800/43; A61K 2800/884

(57) ABSTRACT

A non-film forming composition for colouring fibres, the composition comprising a dispersion of pigment particles in a carrier medium, and a polyelectrolyte having a net charge controllable from a net positive value at a first pH and a neutral or negative net charge at a second different pH, such that the net charge of the polyelectrolyte has a net charge controllable via adjusting the pH of the carrier medium. The polyelectrolyte is adsorbed to the surface of each of the dispersed pigment particles forming pH responsive pigment particles, and wherein the net electrical charge of the pH responsive pigment particles is adjustable via adjusting the pH of the carrier medium.

### FIELD OF THE INVENTION

The present disclosure relates to a composition for colouring fibres, such as keratin fibres, the composition comprising a polyelectrolyte having a net charge controllable from a net positive value at a first pH and a neutral or negative net charge at a second different pH, such that the net charge of the polyelectrolyte complexes has a net charge controllable via adjusting the pH of the carrier medium.

### BACKGROUND OF THE INVENTION

The colouring of natural or synthetic fibres is well known. Dyeing treatments colour natural or synthetic fibres and change their appearance, texture and other physical properties.

Direct dyeing and oxidative dyeing are techniques for colouring hair and other fibres. However, these techniques may have disadvantages such as degradation of the fibres being coloured, irritation to the skin, lack of uniformity and irreversibility. One alternative for dyeing fibres is by the adherence of pigments to the surface of the fibre. An advantage of pigment dyeing, also referred to as pigment colouring, is that it is possible to dye dark fibres without prior bleaching, such as naturally dark coloured hair as the pigment composition generally attaches to the outside of the fibre. Typically, a pigment adhesion dyeing process comprises the provision of a composition comprising a pigment and a film-forming polymer. The composition comprising the pigment and film-forming polymer coats the hair and enables colouring without damage to the fibre. The film-forming polymer is generally a polymer that is capable of forming a continuous, cohesive film attached to the fibre.

However, the present pigment compositions comprising film-forming polymers have drawbacks in that the film-forming polymer inherently alters the physical properties of each fibre and can result in each fibre possessing an artificial feeling. In particular, the fibres may have a rough feeling and lacklustre appearance. Additionally, the fibres may be difficult to comb or brush.

Furthermore, as dyeing of textiles is a water intensive process, and generally irreversible, techniques which enable reversible dyeing of fabric fibres would be advantage. Reversible dyeing compositions and processes may enable reduced total water consumption as they allow textiles to be removed and/or redyed and thereafter reused. Whilst removable colouring is relevant within industrial dyeing of textiles, it is also important within cosmetic hair colouring as simplifying colour removal and subsequent reapplication increases the flexibility of hair colour compositions.

Improved pigment-based fibre dyeing compositions and processes would be advantageous.

### SUMMARY OF THE INVENTION

Accordingly, the present invention preferably seeks to mitigate, alleviate or eliminate one or more of the above-identified deficiencies in the art and disadvantages singly or in any combination and solves at least the above mentioned problems by providing a non-film forming composition for colouring fibres, the composition comprising a dispersion of pigment particles in a carrier medium, and a polyelectrolyte having a net charge controllable from a net positive value at a first pH and a neutral or negative net charge at a second different pH, such that the net charge of the polyelectrolyte has a net charge controllable via adjusting the pH of the carrier medium. The polyelectrolyte is adsorbed to the surface of each of the dispersed pigment particles forming pH responsive pigment particles, and wherein the net electrical charge of the pH responsive pigment particles is adjustable via adjusting the pH of the carrier medium.

A process for treating fibres, such as keratin fibres is also provided.

Further advantageous embodiments are disclosed in the appended and dependent patent claims.

### BRIEF DESCRIPTION OF THE DRAWINGS

FIG. 1 shows a graph of the relationship between pH responsive particle size and the total mass of polymers present in the composition. The graph details a result from experiment 1. The horizontal axis shows the total mass of polymers present in the composition in gram. The vertical axis shows the size of the pH responsive pigment particles in nm.

FIG. 2 shows a graph of the zeta potential curve with respect to pH for pH responsive pigment particles comprising an additional polyelectrolyte, CMC. The graph details a result of experiment 1. The vertical axis shows the zeta potential in mV, the horizontal axis shows pH.

FIG. 3 shows a graph of the zeta potential of a variety of compositions comprising different amounts of an additional polyelectrolyte, CMC. The vertical axis shows the zeta potential in mV, whilst the horizontal axis shows the pH.

### DETAILED DESCRIPTION

The composition for colouring fibres disclosed herein is based on the development of pigment dyeing formulations allowing the colouring of fibres and the latter discoloration, or removal of the colouring from the fibres.

The fibres to be coloured may be for example, natural cellulose based fibres such as cotton, linen, or other cellulose based fibres; keratin fibres, such as human hair or wool. The composition is especially suitable for application to and colouring of keratin fibres, such as human hair.

The composition comprises pigment particles in a carrier medium. The pigment particles are modified via the adsorption of a chemical agent having a modifiable net charge controllable via adjusting the pH of the composition comprising the pigment particle and the net charge modifiable chemical agent. The combined pigment particle and chemical agent having a modifiable net charge controllable via adjusting the pH of the composition is hereinafter referred to as the pH responsive pigment particle. That is the pigment particle has been made pH responsive such that it has a net charge dependent on the pH of the composition, or in general the pH of the environment to which the pH responsive pigment particle is exposed.

To achieve colouring of the fibres the pH responsive pigment particle comprising the pigment particle and the chemical agent adsorbed to the pigment particle must have an opposite charge to the charge of the fibre to be coloured. The term “opposite” refers to the charge having an opposite sign, not the absolute values being the same. Many natural fibres, such as keratin fibres, and in particular human hair, generally have a natural negative charge at a typical treatment pH, such as neutral pH, slightly alkaline pH, or slightly acidic pH. By ensuring the pH responsive pigment particle has a net positive charge, the pH responsive pigment particle, comprising the pigment particle and the adsorbed modifiable chemical agent will be attracted to and adsorbed to the surface of fibres, such as keratin fibres. Thereby the fibres will be coloured by the pigment adsorbing as a result of the electrostatic interaction and van der Waals interactions between the surfaces of the pH responsive pigment particle and fibre.

When removal of the pH responsive pigment from the fibre is desired, the pH responsive pigment particle must be negatively charged, such that the repulsive electrostatic interactions between the pH responsive pigment particle and the fibre desorb the pH responsive pigment particle from the surface of the fibre.

The present composition and processes enable the colouring and decolouring of fibres, such as keratin fibres, without substantially altering the fibres themselves. The composition comprising the pH responsive pigment particles, and in particular, the pH responsive pigment particle does not dissolve or diffuse into the fibres. The pH responsive pigment particle attaches to the outer surface of the fibres and subsequently remains separate from the fibre to which it is attached.

As stated above, the chemical agent having a modifiable net charge is adsorbed to the surface of the pigment particle. Within the composition, the majority, such as greater than 50%, such as greater than 80%, such as greater than 90%, such as greater than 95% of the chemical agent having a modifiable net charge should be adsorbed to the surface of the pigment particle. The chemical agent having a modifiable net charge should generally not be freely dispersed, separate from the pigment particle, within the composition.

The chemical agent having a modifiable net charge may have a net positive charge at a first pH. The chemical agent having a modifiable net charge may have a neutral or net negative charge at a second pH, where the second pH is different to, generally greater than, the first pH. The chemical agent having a modifiable net charge may have a positive net charge at a pH of less than 10, such as less than 9, such as less than 8. The chemical agent having a modifiable net charge may have a neutral or negative charge at a pH greater than about 8, such as greater than about 9, such as greater than about 10.

The chemical agent having a modifiable net charge controllable via adjusting the pH may be a polyelectrolyte. The polyelectrolyte may have a net positive charge at a first pH. The polyelectrolyte may be neutral or carry a net negative charge at a second pH, where the second pH is different to, generally greater than, the first pH. The net charge of the polyelectrolyte may be determined via titration using a particle charge detector.

The polyelectrolyte may be a polycationic polymer, such as a branched or linear organic amine, such as branched polyethyleneimine (PEI). The term polycationic polymer refers to the typical charge of the polyelectrolyte and as discussed below, the charge may be adjusted by adjusting the pH of the carrier medium.

Branched PEI has a dissociation constant (pKₐ) of approximately 9. That is, at up to a pH of approximately 9, the branched PEI has a positive charge. At a pH greater than approximately 9, the branched PEI has a neutral charge.

The branched PEI may be provided at an amount of greater than approximately 0.01% (wt./wt.) with respect to the total weight of the composition. Without wishing to be bound by theory, at an amount of less than 0.01% (wt./wt.) the branched PEI would not be expected to provide any functionality i.e., pH responsiveness to the composition, in particular, would not bind in sufficient amounts to the pigment particles.

The branched PEI may be provided at an amount of less than approximately 10% (wt./wt.) with respect to the total weight of the composition. The branched PEI is advantageously provided at an amount of from about 0.05% (wt./wt.) to about 2% (wt./wt.) with respect to the total weight of the composition, such as from about 0.1% (wt./wt.) to about 1% (wt./wt.), such as from about 0.1% (wt./wt.) to about 0.5% (wt./wt.). The amount of branched PEI may be different with depending on the specific pigment in the composition or the amount of pigment in the concentration. Specifically, with respect to the specific pigment in the composition, the amount of branched PEI in the composition may be adjusted to correspond to the net charge of the selected pigment particle. Pigment particles having themselves each different zeta potential curves, and therein a different net charge at a specific pH, leads to the selection of an appropriate amount of branched PEI.

The composition may comprise a plurality of polyelectrolytes. Each of the plurality of polyelectrolytes being adsorbed to the surface of the pigment particle to form the pH responsive pigment particle. Each or some of the plurality of polyelectrolytes may have different net charges, such as opposite net charges, at the first pH. Each or some of the plurality of polyelectrolytes may have different net charges, such as opposing net charges, at the second pH. Such a combination of a plurality of polyelectrolytes enables the net charge of the pH responsive pigment particle be tuneable via adjusting the ratio of the respective polyelectrolytes. An example of a second polyelectrolyte having a different net charge to the net charge of PEI at the first pH is carboxymethyl cellulose, CMC.

The composition may comprise a weight ratio of pigment to polyelectrolyte of greater than about 1:1, such as greater than about 2:1, and ideally greater than about 3:1, such as about 5:1. The ratio of pigment to branched PEI may be greater than about 1:1, such as greater than about 2:1, and ideally greater than about 3:1, such as about 5:1.

To ensure the pH responsive pigment particles have a controlled and substantially known net surface charge, the composition may be provided with a pH controlling agent. For example, the composition may comprise an acid and/or base selected to ensure the composition has a suitable pH. The experimental section below details the use of hydrochloric acid to provide the composition at a suitable pH for colouring that is, attachment of the pH responsive pigment particles, however, many other acids may be suitable for adjusting the pH of the composition.

The composition for colouring fibres is non-film forming. The term “non-film forming” refers to the pH responsive pigment particles being present in a dispersion within the composition and remaining substantially separate when provided to the fibre to be coloured. The composition does not comprise a film-forming agent. The composition does not comprise a film-forming polymer. Typical film-forming polymers known within the field of fibre colouring are copolymers of vinylpyrrolidone and vinyl acetate monomers, vinylpyrrolidone homopolymers, for example, VP/VA copolymer (or PVP/VA copolymer), PVP. The composition does not comprise the above listed film-forming agents.

To achieve a dispersion of pH responsive pigment particles in the composition, the composition, and specifically the pH responsive pigment particles may be provided with a dispersant. The dispersant ensures that the pH responsive pigment particles do not agglomerate in the composition and when provided to the fibre to be coloured. The dispersant may be for example, a non-ionic, anionic, cationic, or amphoteric surfactant. As disclosed in the experimental section the dispersant may be a non-ionic surfactant. The dispersant is adsorbed to the surface of the pH responsive pigment particle. That is, the dispersant and the polyelectrolyte, such as branched PEI, are adsorbed to the surface of the pigment particle.

The dispersant may be present in the composition at an amount of less than 5% (wt./wt.) with respect to the total weight of the composition, ideally the dispersant is present in an amount of less than 1% (wt./wt.), such as less than 0.5% (wt./wt.), such as less 0.3% (wt./wt.).

The composition may comprise a wetting agent to aid the dispersion of the pH responsive pigment particles within the aqueous carrier medium. The wetting agent is adsorbed to the surface of the pH responsive pigment particles. When the wetting agent is present in the composition, the wetting agent, the chemical agent having a modifiable net charge controllable via adjusting the pH such as the polyelectrolyte, and the dispersant are adsorbed to the surface of each of the pigment particles. The wetting agent may be polyethylene glycol (PEG), such as PEG-200. The wetting agent may be provided at an amount of from about 0.2% (wt./wt.) to about 10% (wt./wt.), such as from about 1% (wt./wt.) to about 5% (wt./wt.), such as from about 1.5% (wt./wt.) to about 4% (wt./wt.), such as from about 2% (wt./wt.) to about 3% (wt./wt.).

The composition may comprise a ratio of pigment to wetting agent of less than about 1:1, such as less than about 1:2, and ideally less than about 1:2.5, such as about 1:3. The composition may comprise a ratio of pigment to PEG of less than about 1:1, such as less than about 1:2, and ideally less than about 1:2.5, such as about 1:3.

As used herein the term pigment refers to any particle colourant comprising or containing pigment material that colours fibres, such as keratin fibres. The pigment particles at least prior to being made pH responsive are substantially non-soluble and poorly dispersible in water. The particular pigment particle, or combination of pigment particles, is selected based on the desired colour to be provided to the fibre, and any additional visual/appearance performance features also desired. The present composition is, however, suitable with various pigments, both organic and inorganic, combinations of organic pigments, combination of inorganic pigments or combinations of organic and inorganic pigments.

The pigment particles may be chosen from the inorganic and organic pigments known within the field of fibre colouring. The pigment particle may be an inorganic or organic pigment particle coated with an additional coating layer, that is, the pigment is coated with an additional layer not relating to the pH responsiveness. The pigment particles can be in the form of a powder or a paste. The pigment may be a mineral pigment, an organic pigment, an elemental metal or a metal oxide, a lake pigment, a composite inorganic-organic pigment such as a nacre, or a mixture thereof.

Inorganic pigments are especially ideal for the pigment particles of the present composition. Inorganic pigments display generally improved resistance to light (both visible and ultraviolet), weather and temperature. Inorganic pigments may be white pigments such as titanium dioxide or zinc oxide, coloured pigments such as red iron oxide. The pigment particles may be selected from metal oxides, hydroxides and oxide hydrates, mixed phase pigments, sulphur-containing silicates, metal sulphides, complex metal cyanides, metal sulphates, chromates and molybdates, alloys, and the elemental metals themselves. The pigment(s) can be selected from stearate coated or siliconized inorganic pigments. The pigment(s) can be selected from the group consisting of titanium dioxide (Ci 77891), black iron oxide (Ci 77499), yellow iron oxide (Ci 77492), red and brown iron oxide (Ci 77491), siliconised iron oxide, manganese violet (Ci 77742), ultramarine (sodium aluminum sulfo silicates, Ci 77007, Pigment Blue 29), chromium oxide hydrate (Ci 77289), Prussian blue (ferric ferrocyanide, Ci 77510), carmine (cochineal), zinc sulfide, barium sulfate, zinc oxide, siliconised titanium dioxide, siliconised zinc sulfide, siliconised zinc oxide, and mixtures thereof. The pigment(s) can be selected from the group consisting of iron oxide, titanium dioxide, mica, borosilicate, and combinations thereof. The pigment can comprise an iron oxide (Fe₂O₃) pigment.

The pigment may be an organic pigment. Organic pigments are organic compounds and have a variety of structures, compositions and therefore colours. The organic pigment be nitroso, nitro, azo, xanthene, quinoline, anthraquinone, phthalocyanin, copper phthalocyanin, copper hexadecachlorophthalocyanine, 2-[(2-Methoxy-4-nitrophenyl)azo]-N-(2-methoxyphenyl)-3-oxobutyramide, metal-complex, isoindolinone, iso indo line, quinacridone, perinone, perylene, diketopyrrolopyrrole, thio indigo, dioxazine, triphenylmethane, dimethylquinacridone and quinophthalone compounds, Azo-dyes, Nonionic azo dyes, Anionic Azo dyes, Cationic azo dyes, Complex forming azo dye, aza annulene dyes, aza analogue of diarylmethane dyes, aza annulene dyes, Nitro-dyes and their pigments, Carbonyl dyes and their pigments (for example, Anthrachinon dyes, indigo), Sulphur dyes, Florescence dyes, Anthracene or Insoluble alkali or earth metal acid dyes. The organic pigment may be any known organic pigment.

The pigment particle may take a variety of forms and shapes depending on the specific pigment and whether it is coated or treated. The pH responsive pigment particle has a substantially similar particle size and shape when compared to the pigment lacking any pH responsiveness. If the pigment particle is spherical, the pigment particle may have a diameter, such as a D50 (vol) diameter, of approximately 0.01 μm to several μm. The pigment particle typically has a D50 (vol) diameter of approximately 0.05 μm to 1 μm, such as from about 0.05 μm to about 0.5 μm. If the pigment particle is spherical, the pH responsive pigment particle, that is the pigment particle with at least the chemical agent having a modifiable net charge controllable via adjusting the pH of the composition adsorbed to the surface of the pigment particle, may have a diameter of approximately 0.01 μm to several μm, such as from about 0.05 μm to about 1 μm.

Different pigment particles may be combined to achieve improved visual appearance of the coloured fibres. For example, a combination of at least two different pigment materials to form a combined pigment mixture. The combined pigment mixture may provide improved reflective, refractive and light transmitting properties to the coloured fibre.

The zeta potential of the pigment particle may change depending on the pH of the carrier medium in which it is present. The pigment particle may have a zeta potential less than −10 mV from about pH 4 to about pH 8. The pigment particle may have a zeta potential of from about 0 to about-40 mV from about pH 4 to about pH 9. The pigment particle may have a zeta potential of about 35 mV to about 0 mV from about pH 4 to about pH 9, such as to about pH 8. The zeta potential of the pigment may be measured as described in the experimental section.

The composition may comprise the pigment particles at an amount of from about 0.01% (wt./wt.) to about 20% (wt./wt.) with respect to the total weight of the composition, such as from about 0.1% (wt./wt.) to about 5% (wt./wt.).

The carrier medium of the composition may be an aqueous carrier medium. The composition may be considered an aqueous composition comprising the pH responsive pigment particles. The composition may comprise the carrier medium at an amount of from about 50% (wt./wt.) to about 99% (wt./wt.) with respect to the total weight of the composition, such as from about 80% (wt./wt.) to about 99% (wt./wt.), such as from about 93% (wt./wt.) to about 98% (wt./wt.) carrier medium. The carrier medium may be water.

The composition may be a liquid dispersion, a gel dispersion, a cream dispersion or any other form which is suitable for applying to the fibres to treated.

The composition is a ready-to-use composition, the term “ready-to-use” refers to the composition being suitable for direct application to the fibres to be treated. The composition is provided in a form which is suitable for use directly and is does not result from the mixing of, for example, several separate components in conjunction with application to the fibres to be treated.

The composition may optionally include, or optionally exclude, one or more miscellaneous ingredients. Miscellaneous ingredients are ingredients that are compatible with the cosmetic compositions and do not disrupt or materially affect the attachment of pH responsive pigment particles of the compositions. Nonlimiting examples of miscellaneous ingredients include preservatives, fragrances, antioxidants, flavonoids, vitamins, botanical extracts, UV filtering agents, proteins, protein hydrolysates and/or isolates.

The total amount of the one or more miscellaneous ingredients, if present, will vary. Nonetheless, in various embodiments, the composition may comprise from about 0.001% (wt./wt.) to about 10% (wt./wt.) of one or more miscellaneous ingredients, with respect to the total weight of the composition.

To colour the fibres, the composition may be applied to the fibres, such as keratin fibres, according to the following process:
the composition is applied to the fibres, the composition may be applied via direct application via a brush, spray etc. or washed-in via for example, a shampoo comprising the present composition; and
the composition is left to act on the fibres for a period form about 30 seconds to about 48 hours, preferably without rinsing. The composition may be optionally dried, such as via blow drying.

The pH responsive pigment particles are thereby attracted via electrostatic interaction and van der Walls forces to the fibres and remain attached to the fibres.

To remove the composition from the fibres the following process may be used:
an alkaline medium is applied to the fibres, if the fibres are hair fibres, the alkaline medium may be a shampoo having a pH of greater than about 8; the alkaline medium alters the net surface charge of the pH responsive pigment particles such that the pH responsive pigment particles are electrostatically repelled by the fibres, the pH modified pigment particles are thereafter detached from the fibres. The alkaline medium may additionally and advantageously comprise at least one surfactant known within the field. The at least one surfactant may be a non-ionic, anionic, cationic, or amphoteric surfactant. The at least one surfactant may be for example, sodium lauryl sulphate (SDS/SLS). The alkaline medium may comprise at least two surfactants such as a non-ionic surfactant and an anionic surfactant.

The present composition is ideally what is known within the field as a “leave-in” composition whereby a post-application rinsing step is not necessary for activating the colour. As the pH responsive pigment particles are attracted to the fibres via electrostatic interaction and the pH responsive pigment particles provide an external coating to the fibre the colour is provided to the fibre directly on application of the composition.

The composition may be applied to wet or dry fibres. The fibres, such as keratin fibres, may be washed prior to application of the fibres. The composition may be applied to the fibres at room temperature.

### EXPERIMENTAL SECTION

### Experiment 1

### Preparation of Composition Comprising pH Responsive Pigment Particles

To prepare the composition, 0.2 g of the branched polyelectrolyte PEI was dissolved in 40 mL of water. Then 0.3 g of pigment (PV23, hydrophobic violet pigment) was added, and the solution was stirred for 2 minutes. 1 mL of PEG-200 was then added to act as a wetting agent and to disperse the pigment in water and the solution was shaken and then sonicated using a sonicator for 1 minute at 50% amplitude. 0.1 g of the non-ionic surfactant C₁₁E₈ (Ethylan® 1008, Nouryon) are subsequently added and then the pH is adjusted to 6.8 with hypophosphorous acid. The dispersion was sonicated again for 30 seconds at 50% amplitude. After this first sonication, 0.2 g of an amphiphilic polymer with sulphated polar heads (Tego® 656, Evonik Operations GmbH) was added. The pH was adjusted to 12 by the addition of sodium hydroxide, NaOH. The composition was sonicated again, to allow the pH responsive pigment particles to disperse and the polymers (PEI, PEG, Tego) to be adsorbed on the surface of the pigment particles.

### Effect of Amount of Polymer on Particle Size

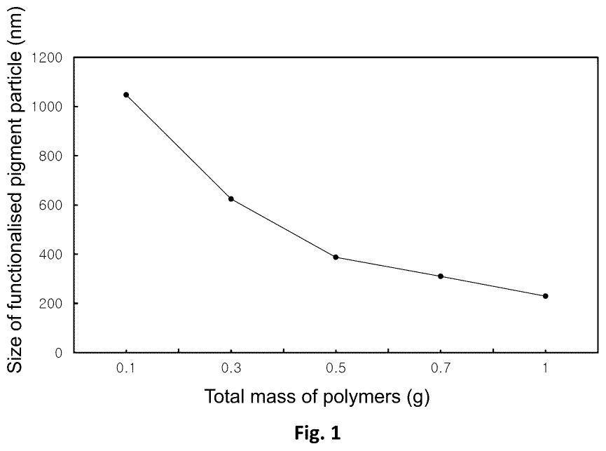

To determine the resulting particle size within the composition. An experiment consisting of measuring the particle size by Dynamic Light Scattering as a function of the total mass of PEI, Tego 656, and Ethylan 1008 was performed. The mass proportions between the three respective components was maintained. Several compositions were made with a total mass of PEI, Tego 656, Ethylan 1008 of 0.1 g, 0.3 g, 0.5 g, 0.7 g, and 1 g. The particle size was measured by DLS at a pH of approximately 7. The dynamic light scattering measurement is performed with a submicron particle analyser (Beckman Coulter, N4 Plus). The results are shown in FIG. 1.

As can be seen in FIG. 1, as the total mass of polymers increases, the size of the pigment particle decreases. That is, an increase in polymer amount results in a decrease in particle size. A smaller particle size increases colloidal stability and results in improved and more effective colouring as the specific surface area of the particles is increased with respect to the total particle amount in the composition. Visual inspection confirmed the colloidal stability of the compositions having smaller particle sizes. The composition with a total polymer mass of 0.1 g at a pH of 12 showed clear sedimentation of the particles due to the larger particle size. The composition comprising a total polymer mass of 0.5 g did not settle and was stable at a pH range from 4 to 12.

### Determining Isoelectric Point of pH Responsive Pigment Particles

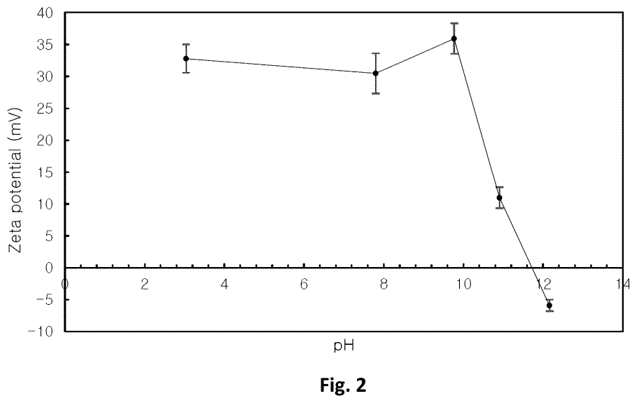

The zeta potential of the composition comprising 0.5 g of total polymer mass as a function of pH was measured by a ZetaPALS system (Brookhaven Instruments, USA). An electrode cleaned with ethanol and water is placed in a receptacle comprising 1.4 mL of the composition which allowed the measurement of electrophoretic mobility. The results are shown in FIG. 2.

According to FIG. 2, it is observed that at acidic pH, the zeta potential was around +35 mV, which was high enough to have a stable colloidal dispersion and good attachment to negatively charged fibres.

### Experiment 2: Hair Colouring with Composition Comprising PEI, Tego and CMC for Improved Decolouration

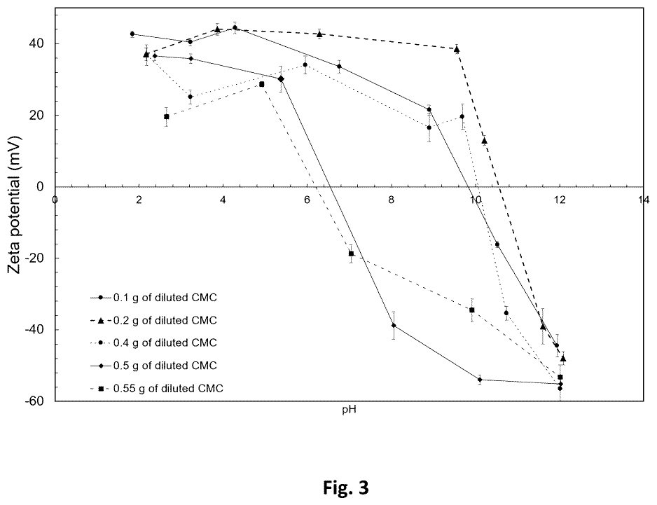

In order to provide a composition having a decreased zeta potential (more negative) at higher pH, the polyelectrolyte carboxymethyl cellulose (CMC) was provided to the composition described in experiment 1. The composition comprised 0.2 g of PEI, 0.2 g of Tego, and the remaining components of the composition were unchanged. CMC was provided at varying amounts and the zeta potential as a function of pH was determined for each of the compositions comprising the CMC. The results are shown in FIG. 3.

As can be seen in FIG. 3, the provision of the additional polyelectrolyte CMC, decreases the zeta potential of the pH responsive pigment particles when compared to the results shown in FIG. 2. At acidic pH, the zeta potential was positive and high, around +35 mV. At basic pH the zeta potential reached values approaching −50 mV. With a composition comprising CMC, which decreases the zeta potential at higher pH, improved decolouring can be achieved as the negative charge of the pH responsive pigment particle is desorbed and repelled from the surface of the negatively charged fibre.

The composition according to experiment 1 with 0.2 g of PEI, 0.2 g of Tego, and 0.2 g of CMC was diluted fivefold with water. At a pH of 4.88 no sedimentation occurred, i.e., the dispersion was stable. The diluted composition was applied to blond hair. The hair displayed vibrant colouring and was fast and resistant to mechanical abrasion.

The hair coloured with the diluted composition was thereafter decoloured. The hair was decoloured at a pH of 12.21 with 1% SDS and 1% Ethylan 1008. The decolouration was effective and the pH responsive pigment particles were removed from the hair. During hair decolouring the hair was rubbed to provide some mechanical abrasion.

### Experiment 3: Alternative Colouring Composition

A composition not comprising the amphiphilic polymer (Tego 656) nor the additional polyelectrolyte (CMC) was prepared.

The composition comprised pigment, PEI, a dispersant (non-ionic surfactant Ethylan® 1008, Nouryon), a wetting agent (PEG-200) and water as detailed in the following table.

| Component | Amount | |
|---|---|---|
| Pigment | 150 | mg |
| Dispersant (Ethylan 1008) | 50 | mg |
| Wetting agent (PEG-200) | 0.5 | ml |
| Water | 20 | ml |
| PEI (concentration: 5% wt./wt.) | 0.6 | ml |

The composition was prepared as follows: 150 mg of pigment was added to a 30 ml vial. 0.6 ml of PEI solution was added to the pigment. 50 mg of the dispersant was added to the mixture. 0.5 ml of the wetting agent was added to the mixture. 20 ml of water was added to the mixture to form an aqueous dispersion of pH responsive pigment particles, forming the composition comprising pH responsive pigment particles. The dispersion was thereafter sonicated for 30 seconds to ensure particle separation within the dispersion. The pH of the dispersion was lowered to about 5.5 via the addition of hydrochloric acid. The particle size and zeta potential of composition comprising the pH responsive pigment particles were determined via DLS and the ZetaPALS system as described for experiment 1.

The composition was thereafter applied to hair swatches and left for about 10 minutes. The hair samples were inspected. The coloured hair showed high colour intensity and uniformity. The hair felt very similar to a control hair swatch which had not been coloured. The coloured hair was immersed in water for 30 mins to determine colour fastness. The hair was subsequently wiped with paper towel. The appearance of the coloured hair after immersion in water was similar to the appearance before. Additionally, there was little to no colour present on the paper towel indicating the pH responsive pigment particles had adhered satisfactorily to the hair fibres.

### Experiment 4: Application and Removal of Colouring Composition to Cotton Fibres

To determine the effectiveness of colouring fibres in addition to human hair the composition of experiment 2 was applied to natural cotton fibres. The natural cotton fibres have a natural net negative surface charge. The composition according to experiment 2 was diluted by a factor of 5 with water to ensure the colloidal stability of the composition. A cotton fabric sample was immersed in the dilute composition for 5 minutes. The pH of the composition on immersion was 6.94 (that is, about 7). Intense and homogenous colouring of the cotton fabric sample was observed.

To determine the effectiveness of decolouring the cotton fabric sample was cut into five pieces and each piece was immersed in a different solution having a known pH for 5 minutes at room temperature (except for 12.01, as described below). The solutions were stirred during the 5 minutes of decolouration. The pH values of the different solutions were 2.9, 8.1, 12.01, 12.07 and 12.17. The solution having a pH of 12.01 was warmed to 60° C. to determine the effect of elevated temperature on decolouration. The solution having a pH of 12.07 was placed in an ultrasonic bath at room temperature.

As expected, the pieces of fabric in the solutions of pH 2.9 and 8.1 displayed no decolouration. The pH responsive pigment particles remained fixed to the cotton fibres. Decolouration occurred for the solutions at pH 12.01, 12.07 and 12.17. No increased decolouration occurred for the fabric piece subjected to elevated temperature. The fabric piece subjected to the ultrasonic bath displayed slightly improved decolouration. To validate this effect the experiment above was repeated with a larger piece of fabric and ultrasonic agitation was carried out locally on specific areas of the fabric. The results showed that local application of ultrasonic agitation to specific areas of the fabric resulted in improved decolouration to such areas.

The initial cotton, the cotton coloured at a pH of 6.94 for 5 minutes with stirring and the cotton decoloured at room temperature in the ultrasonic bath, were analysed using a portable spectrometer to determine the colour of the textile in the CIE Lab coordinate system. The results table below shows the different values L*, a* and b* of the original, coloured and decoloured cotton. Three quantities characterize the colours: the lightness L* derives from the luminance of the surface, the parameter a* represent the value on a green to red axis and the parameter b* represents the value on a blue to yellow axis. Two measurements were taken at different places on the cotton samples.

| Sample | L* | a* | b* |
|---|---|---|---|
| Initial non-coloured cotton | 95.53 | −0.22 | −1.31 |
| | 95.58 | −0.18 | −1.76 |
| Coloured cotton | 51.50 | 19.24 | −30.34 |
| | 51.52 | 19.34 | −30.57 |
| Decoloured cotton | 79.09 | 11.58 | −20.11 |
| | 79.13 | 11.73 | −20.60 |

### Experiment 5: Colouring Wool Fibres with Composition

A composition according was prepared according to experiment 3. The pH of the dispersion of pH responsive pigment particles was adjusted to pH 6.4 in preparation of the application step. The composition was applied to wool and left to colour the wool for 10 minutes. The pigment colour was bright and intense on the wool. After colouring, the coloured wool sample was rubbed with paper towel. There was some minor colour bleed into the paper towel on rubbing. The coloured wool sample was rinsed with water. There was no significant difference in colour intensity after rinsing the wool with water. Experiment 5 showed that the composition is suitable for colouring wool fibres.

Although, the present invention has been described above with reference to specific embodiments, it is not intended to be limited to the specific form set forth herein. Rather, the invention is limited only by the accompanying claims.

In the claims, the term “comprises/comprising” does not exclude the presence of other elements or steps. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

We claim:

1-23. (canceled)

24. A non-film forming composition for colouring fibres, the composition comprising:
a dispersion of pigment particles in a carrier medium, and
a polyelectrolyte having a net charge controllable from a net positive value at a first pH and a neutral or negative net charge at a second different pH, such that the net charge of the polyelectrolyte has a net charge controllable via adjusting the pH of the carrier medium,
wherein, the polyelectrolyte is adsorbed to the surface of the dispersed pigment particles forming pH responsive pigment particles, and wherein the net electrical charge of the pH responsive pigment particles is adjustable via adjusting the pH of the carrier medium.

25. The non-film forming composition according to claim 24, wherein the pH responsive pigment particles adsorb to the surface of the fibres at the first pH, and wherein the pH responsive pigment particles desorb from the surface of the fibre at the second pH.

26. The non-film forming composition according to claim 24, wherein the composition is for colouring keratin fibres.

27. The non-film forming composition according to claim 24, wherein the polyelectrolyte is a branched or linear organic amine.

28. The non-film forming composition according to claim 24, wherein the polyelectrolyte is branched or linear polyethyleneimine.

29. The non-film forming composition according to claim 24, wherein the first pH is substantially less than the second pH.

30. The non-film forming composition according to claim 24, wherein the first pH is less than about 8, such that the polyelectrolyte possesses a net positive charge at a pH of less than about 8.

31. The non-film forming composition according to claim 24, wherein the pH responsive pigment particles possess a net positive charge at the first pH.

32. The non-film forming composition according to claim 24, wherein the second pH is greater than about 7, such that the polyelectrolyte possesses a net negative or neutral charge at a pH of greater than about 7.

33. The non-film forming composition according to claim 24, wherein the pH responsive pigment particles possess a net negative or neutral charge at the second pH.

34. The non-film forming composition according to claim 24, wherein the pH responsive particles possess an opposite net electric charge to fibres to be coloured.

35. The non-film forming composition according to claim 24, wherein the polyelectrolyte is a first polyelectrolyte and wherein the composition comprises a second polyelectrolyte, wherein both the first and second polyelectrolytes are adsorbed to the surface of the dispersed pigment particles, and wherein the second polyelectrolyte has a different net charge with respect to the first polyelectrolyte at the first pH, and optionally a different net charge with respect to the first polyelectrolyte at the second pH, such that net charge of the pH responsive pigment particle is tuneable via adjusting the ratio of the first polyelectrolyte to the second polyelectrolyte.

36. The non-film forming composition according to claim 24, wherein the pigment particles are organic or inorganic pigment particles.

37. The non-film forming composition according to claim 24, wherein the composition comprises a dispersant adsorbed to the pigment particles.

38. The non-film forming composition according to claim 24, wherein the composition comprises a wetting agent adsorbed to the pigment particles.

39. The non-film forming composition according to claim 24, wherein the composition comprises at least 0.1% (wt./wt.) polyelectrolyte.

40. The non-film forming composition according to claim 24, wherein the composition comprises less than about 10% (wt./wt.) polyelectrolyte.

41. The non-film forming composition according to claim 24, wherein the composition comprises a ratio of pigment particles in the composition to polyelectrolyte at a weight ratio of greater than about 1:1.

42. A process for treating fibres, the process comprising:
providing a composition, the composition comprising:
a dispersion of pigment particles in a carrier medium, and
a polyelectrolyte having a net charge controllable from a net positive value at a first pH and a neutral or negative net charge at a second different pH, such that the net charge of the polyelectrolyte has a net charge controllable via adjusting the pH of the carrier medium, wherein, the polyelectrolyte is adsorbed to the surface of each of the dispersed pigment particles forming pH responsive pigment particles, and wherein the net electrical charge of the pH responsive pigment particles is adjustable via adjusting the pH of the carrier medium; and,
applying the composition to the fibres to be treated, wherein the pH of the composition is selected such that the total net charge of the pH responsive pigment particles is opposite to the net surface charge of the fibres to be treated, and thereby colouring the fibres.

43. The process according to claim 42, wherein the process comprises:
providing a second composition having a pH of such that the total net charge of the pH responsive pigment particles is opposite to the charge of the pH responsive pigment particle on application to the fibre, and thereby
removing the pH responsive pigment particles from the fibres, thereby decolouring the fibres.

44. The process according to claim 42, wherein the fibres are human hair.

45. The process according to claim 42, wherein the first composition is provided at a pH substantially lower than 7.

46. The process according to claim 43, wherein the second composition is provided at a pH of substantially greater than 7.
